(19) United States
(12) Patent Application Publication
(10) Pub. No.: US 2003/0211062 A1
(43) Pub. Date: Nov. 13, 2003

(54) Anhydrous skin cleaners

(76) Inventors: Karl Laden, Haifa (IL); Harry Brand, Haifa (IL)

Correspondence Address: OLSON & HIERL, LTD., 20 NORTH WACKER DRIVE, 36TH FLOOR, CHICAGO, IL, 60606, US

(21) Appl. No.: 10/333,671

(22) PCT Filed: May 7, 2001
(86) PCT No.: PCT/US01/14570

(51) Int. Cl. A61K 7/06

(52) U.S. Cl. 424/70.1

(57) ABSTRACT

Anhydrous skin cleansers, preferably in the form of gels, comprise water-immiscible emollient oil; oil-gelling agent that is water-insoluble and oil-insoluble; emulsifying agent that forms an emulsion in situ on the skin when the skin cleanser is gradually contacted with a relatively small amount of water during use; and water-soluble, substantially crystalline abrasive material that is insoluble in the anhydrous skin cleanser vehicle. The anhydrous skin cleanser gels remove oily and water-soluble soils from the skin in a cosmetically aesthetic manner. Preferred skin cleanser gels form cleanser emulsions in situ, on the skin during use that can be removed from the skin with water.

### CROSS-REFERENCE TO A RELATED APPLICATION

This application claims the benefit of U.S. Provisional Application Serial No. 60/202,409 filed on May 8, 2000.

### TECHNICAL FIELD OF THE INVENTION

This invention relates generally to cosmetic preparations for application to the skin and in particular to anhydrous preparations for cleansing the skin.

### BACKGROUND OF THE INVENTION

The formulation of skin cleansers and polishers present numerous challenges for the cosmetic chemist. In order to cleanse the skin, a combination of skin oils, sebum, sweat, makeup and environmental dirt must be removed. In many cases the use of an abrasive additive is also required to enhance the cleaning. These abrasives can also serve as a polishing agent by removing rough skin flakes and thereby smoothing the skin surface. All of these functions must be performed while at the same time leaving the skin in good condition.

It is well known that many of the surface-active agents (such as soaps and synthetic detergent bars) normally used to clean skin can leave the skin overly dry. They can be too aggressive in removing the oily soils off the skin and in so doing, remove vital skin oils and water-soluble natural humectants necessary for good skin health. They are also not very efficient in removing many types of makeup residues.

The use of aqueous based skin cleansers can remove water-soluble soils and easily solubilized oils, but are often less efficient in removing greasy soils and makeup ingredients that are difficult to solubilize off the skin surface. They also can dry out the skin.

Oil-in-water emulsion cleansers, while milder on the skin, are generally not very good in removing many cosmetic and makeup residues that contain water-insoluble waxes and other difficult to dissolve generally water-insoluble organic residues. Water-in-oil emulsions tend to be much more effective than oil-in-water emulsions in solubilizing and removing oily soils and greasy residues, but are generally inferior in cleansing water-soluble soils. Moreover, water-in-oil emulsions sometimes can be irritating to the skin and, if not vigorously rubbed or washed off, can often leave an unwanted oily residue on the skin.

Prior attempts to improve the efficacy of aqueous skin cleanser systems have been made by the inclusion of abrasive materials. Abrasive materials can significantly contribute to the cleaning ability by removing superficial skin cells thereby smoothing or polishing the skin surface. However, the choice of the right particle size for these abrasives creates a dilemma. A coarse particle size may provide some measure of increased abrasive cleaning power but also increases the risk of scratching the skin surface. A fine particle size may provide some smoothing and polishing of the skin surface but generally is less effective in soil removal.

Prior attempts have been made to overcome the foregoing problems of aqueous emulsions by employing anhydrous skin cleansers. Anhydrous skin cleansers, sometimes called waterless cleansers, typically contain high concentrations of water-insoluble solvents, which makes them generally effective at removing oily soils from the skin, but less effective in removing water-soluble soils. Further, anhydrous skin cleansers typically are not cosmetically elegant having a heavy, greasy feel making them unappealing to the touch and are not easily removed from the skin. They generally must be wiped off with toweling, leaving the skin feeling greasy or washed off with strong soap, leaving the skin feeling harsh and dry.

There is an ongoing need and desire, therefore, for a cosmetically elegant, anhydrous skin cleanser, capable of removing greasy and non-greasy soils and rough, dead skin debris without scratching or irritating the skin, and yet be removable with water, leaving the skin in a smooth, moisturized condition.

The present anhydrous skin cleansers provide skin cleansing and polishing in a cosmetically aesthetic vehicle while, at the same time, leaving the skin exceptionally soft, smooth and moisturized.

### SUMMARY OF THE INVENTION

The present invention relates to substantially anhydrous skin cleansing and polishing formulations, preferably in gel form, and a method for using these formulations in a manner that enhances their effectiveness.

Preferred skin cleanser composition embodiments of this invention are substantially anhydrous gels comprising: a) at least one water-immiscible cosmetic emollient oil, the cosmetic emollient oil comprising a major portion of the formulation; b) at least one oil-gelling agent that is both water-insoluble and oil-insoluble; c) at least one emulsifying agent capable of forming an emulsion, in situ, on the skin when a small amount of water is added gradually to the substantially anhydrous formulation during use; and d) a substantially crystalline, water-soluble, abrasive material that is substantially insoluble in the substantially anhydrous gel vehicle of the formulation.

Surprisingly, the present substantially anhydrous skin cleansers have been found to work particularly well when the skin cleanser is first applied to substantially dry, soiled skin and rubbed or massaged into the soiled skin to coat the skin, the so-coated skin portion is then gradually contacted with a small amount of water, preferably of not more than about two parts by weight of the amount of skin cleanser applied, and the rubbing, massaging action is resumed until the skin cleanser forms a cleanser emulsion in situ on the skin site and continued until the abrasive material substantially dissolves. The resulting cleanser emulsion can then be rinsed off the skin with water.

Preferred anhydrous skin cleanser formulations comprise, on a total composition weight basis: a) about 30% to about 90% of cosmetic emollient oil; b) about 1% to about 20% of oil-gelling agent that is insoluble in both water and oil; c) about 1% to about 15% of emulsifying agent that readily forms an emulsion in situ on the skin when a small quantity of water is added gradually to the anhydrous formulation during use; and about 1% to about 60% of water-soluble, substantially crystalline abrasive material that is substantially insoluble in the anhydrous skin cleanser vehicle.

The inventive anhydrous skin cleansers beneficially achieve excellent cleaning of all types of skin residues, remove rough skin cells, polish the skin surface and leave the skin with a very soft smooth feel. Surprisingly, the anhydrous skin cleanser compositions remove oily and greasy soils as well as water-soluble soils and are easy to remove with water while remaining in a cosmetically aesthetic form. Another advantage is that the abrasive material provides sufficient soil removing abrasiveness without irritating or scratching the skin. Thus, the beneficial and desirable cleansing efficacy normally attributed to both anhydrous cleansers, water-in-oil emulsions and oil-in-water emulsion are achieved in a single formulation vehicle without the attendant negative afterfeel on the skin typically associated in the prior art with the usage of the individual foregoing cleanser vehicles.

### DESCRIPTION OF THE PREFERRED EMBODIMENT

The term “substantially anhydrous”, and “anhydrous” are used interchangeably herein to refer to skin cleanser composition having not more than about 5 weight percent free water content on a total composition weight basis. With reference to substantially crystalline abrasive material, the term “free water content” does not include water of crystallization. The term “substantially anhydrous”, as applied to abrasive materials, denotes water-soluble, particulate abrasive materials having a particle size sufficient to abrasively remove soil and cellular debris from the skin without scratching or irritating the skin. The term “substantially dry” as applied to skin as used herein means that the skin has not been topically moistened or wetted with externally applied water and does not include moisture naturally present in the skin or on the skin from perspiration.

The novel anhydrous skin cleanser is preferably in the form of a non-runny gel comprising, on a total compositions weight basis:

| Ingredients | | Weight percent |
|---|---|---|
| a) | Water-immiscible | about 30 to about 90 |
| | emollient oil. | |
| b) | Oil-gelling agent that is | about 1 to about 20 |
| | both water-insoluble and | |
| | oil-insoluble. | |
| c) | Emulsifying agent | about 1 to about 15 |
| | capable of forming an | |
| | emulsion, in situ, | |
| | on the skin when a | |
| | small amount of water | |
| | is added to the anhydrous | |
| | formulation during use. | |
| d) | Substantially crystalline | about 1 to about 60 |
| | abrasive material that is | |
| | water-soluble and substantially | |
| | insoluble in the anhydrous | |
| | formulation vehicle. | |
| e) | Cosmetic adjuvants | optional |
| | (e.g. fragrance, color, | |
| | preservatives, antioxidants, | |
| | keratolytic agents, pH adjusting | |
| | agents and the like). | |

With regard to preferred ingredients, those skilled in the cosmetic formulation arts are familiar with cosmetically acceptable ingredients and optional cosmetic adjuvant ingredients conventionally employed and commercially available from numerous sources. A comprehensive listing of cosmetic ingredients and suppliers can be found in the International Cosmetic Ingredient Dictionary, (“INCI Dictionary”) Sixth Edition, published by the Cosmetic, Toiletry, and Fragrance Association, Washington, D.C. (1995). For convenience, ingredients generally will be referred to by the industry recognized standardized designations commonly referred to as “INCI” names given them in the INCI Dictionary, the relevant disclosures of which are incorporated herein by reference. An extensive listing of cosmetic ingredients can also be found in Cosmetics & Toiletries® Cosmetic Bench Reference 2001, 115(13), published by Allured Publishing Corporation, Carol Stream, Ill. (2001), (hereafter referred to as the “CBR”) the relevant portions of which are incorporated herein by reference.

Preferably one or more water-immiscible emollient oils typically used in cosmetic formulations are employed. On a total composition weight basis, a suitable weight amount of water-immiscible emollient oil preferably is in the range of about 30 to about 90 weight percent, more preferably in the range of about 40 to about 85 weight percent. Examples of cosmetically acceptable water-immiscible oils include, without being limited thereto, linear and branched chain aliphatic hydrocarbons, animal derived oils, vegetable derived oils, silicone oils, esters of fatty acids having at least 7 carbon atoms in the fatty chain, C₇-C₁₈ aliphatic and aromatic alcohols and mixtures thereof.

Exemplary water-immiscible linear aliphatic hydrocarbons are preferably petroleum derived hydrocarbons including, but not limited, to mineral oil, paraffin oil, petroleum jelly, and the like. Exemplary branched chain aliphatic hydrocarbons include, but are not limited thereto, isododecane, isohexadecane and the like.

Exemplary water-immiscible animal derived oils, include, but are not limited to, lanolin oil, mink oil, fish oils, milk fat and the like. Exemplary water-immiscible vegetable derived oils include, but are not limited to, sweet almond oil, sunflower oil, coconut oil, babassu oil, palm oil, palm kernel oil, corn oil, peanut oil, grape seed oil, safflower oil, poppy seed oil, sesame seed oil, wheat germ oil, avocado oil, olive oil, soybean oil, meadowfoam seed oil, jojoba oil and the like and mixtures thereof.

Exemplary water-immiscible silicone oils include dimethicone, cyclomethicone and the like;

Exemplary water-immiscible emollient esters include, but are not limited to, isopropyl myristate, isopropyl palmitate, octyl palmitate, octyl stearate, isocetyl stearate, caprylic/capric triglycerides, decyl oleate and mixtures thereof.

Exemplary C₇-C₁₈ aliphatic and aromatic alcohols include, but are not limited to, heptanol, octanol and the like.

Preferably one or more oil-gelling agents that are insoluble at 25° C. in water and in oil are employed. On a total composition weight basis, a suitable weight amount of oil-gelling agent is in the range of about 1 to about 20 weight percent, more preferably in the range of about 2 to about 10 weight percent. The final gelled formulations preferably have a viscosity in the range of about 5 to about 250 poise. Examples of gelling agents that can be used include, without being limited thereto, clays and organically modified clays, silicas, soaps of fatty acids having at least 11 carbon atoms in the fatty chain and a Group IA, IIA or IIIA metal ion, aluminum/magnesium hydroxide stearate gels and mixtures thereof.

Exemplary oil-gelling clays and organically modified clays preferably include, without being limited thereto, bentonites, hectorites, montmorillonites, organophilic clays, such as Stearalkonium hectorite, Quaternium-18 hectorite, Quaternium-18 bentonite and the like. Organically modified clays are frequently commercially provided in pre-gelled form, sometimes called mastergels, which include a relatively low quantity of a wetting or swelling agent such as propylene carbonate. Examplary pre-gelled organically modified clays include, without being limited thereto, mastergels sold under the trade names of Miglyol (Condea), Tixogel (Sud-Chemie) and Bentone Gel (Rheox).

Exemplary silicas preferably include, without being limited thereto, finely divided silicas that are colloidal in nature having a particle size in the range of about 0.005 to 0.05 microns; magnesium aluminum silicate and the like. Suitable colloidal silicas are commercially sold under the trade names of Cab-O-Sil (Cabot Corp.) and Aerosil (Degussa).

Exemplary soaps are preferably non-toxic Group IA, IIA and IIIA metal salts of fatty acids having at least 11 carbon atoms, preferably at least 13 carbon atoms, more preferably at least 17 carbon atoms in the fatty chain. Group IA metal soaps are preferably sodium and potassium salts; exemplary Group IIA metal soaps are magnesium salts, and Group IIIA metal soaps are preferably aluminum salts. Particularly preferred, without being limited thereto, are magnesium stearate, aluminum stearates.

Useful aluminum/magnesium hydroxide stearate gel are sold under the trade name of Giuligels (BK-Giulini).

Emulsifiers capable of forming an emulsion, in situ, on the skin during use, when the skin cleanser gel is contacted gradually with water, are employed on a total composition weight basis in an amount preferably in the range of about 1% to about 15%, more preferably in the range of about 2% to about 10%. It is preferred to use a mixture of at least two emulsifiers, one having a relatively low HLB value preferably of not more than 8, more preferably in the range of about 3 to about 7, and one having a relatively high HLB of preferably at least 8, more preferably in the range of about 10 to about 19 Those skilled in the cosmetic emulsion arts will understand that the term “HLB” refers to the well known calculated Hydrophile-Lipophile-Balance value assigned to emulsifiers, most commonly nonionic emulsifiers, relating to the water solubility of the emulsifier. A discussion of the calculation of HLB values can be found in Chapter 10 of Rieger (Ed), Harry's Cosmeticology, 8th Edition, Chemical Publishing Co., Inc. New York, N.Y. (2000), the relevant disclosures of which are incorporated herein by reference. An extensive listing of emulsifiers and HLB values can also be found in the CBR issue of Cosmetics & Toiletries® magazine described above, the relevant portions of which are incorporated herein by reference.

It was surprisingly found that the anhydrous skin cleansers of the present invention readily formed an emulsion when the anhydrous skin cleanser gel vehicle was contacted with a relatively low amount of water of not more than about two parts of water based on the total weight of composition. Without being bound by any theory, it is believed that a low HLB emulsifier promotes the uptake of low amounts of water by the gelled oil vehicle to first form a water-in-oil emulsion in situ on the skin when the skin cleanser gel is contacted with such a relatively low amount of water during use. It is also believed that a relatively high HLB emulsifier facilitates the rinsing of the product from the skin by further promoting the formation of a multiphase emulsion or inversion to an oil-in-water emulsion in situ. Thus, the skin cleansing gel of this invention beneficially retains the oily soil removal efficacy usually attributable to water-in-oil emulsions without leaving an unwanted oily residue on the skin.

Exemplary emulsifying agents include, without being limited thereto, ethoxylated carboxylic acids, ethoxylated glycerides, glycol esters and derivatives thereof, monoglycerides, polyglyceryl esters, polyhydric alcohol esters and ethers, sorbitan/sorbitol esters, triesters of phosphoric acid, ethoxylated fatty alcohols, propoxylated polyoxyethylene (POE) ethers and the like and mixtures thereof. Particularly preferred are glyceryl stearate, PEG-100 stearate, sorbitan stearate, PEG-40 stearate, steareth-2, steareth-20, steareth-100, polysorbate-20, laureth-1, laureth-23 and the like. A description of commercially available ester and ether emulsifiers and suppliers thereof can be found in Chapter IV of Rieger, Surfactant Encyclopedia, 2nd Edition, published by Cosmetics & Toiletries® magazine, Allured Publishing Corporation, Carol Stream, Ill. (1996), the relevant disclosures of which are incorporated herein by reference.

One or more substantially crystalline, water-soluble abrasive materials are employed, preferably having sufficient water solubility such that at least 20 parts by weight of the abrasive will dissolve in 100 parts by weight of water at 20° C., and which are insoluble in the anhydrous skin cleanser formulation vehicle. The particle size of the abrasive crystalline material should be sufficiently large to serve as a scrubbing agent against the skin to remove dirt and cellular debris, such as rough dead skin cells, and yet not so large as to scratch or irritate the skin. A preferred particle size in the range of about 50 to about 1000 microns has been found useful for removing soil and dead skin cells. This particle size range also permits the rapid dissolution of the abrasive when water is added to the formulation during usage on the skin. Preferably, on a total composition weight basis, an amount of substantially crystalline abrasive material employed is in the range of about 1% to about 60%, more preferably in the range of about 2% to about 50%.

Examplary water-soluble/vehicle-insoluble, substantially crystalline abrasive materials include, without being limited thereto, Dead Sea salt, salts of Group IA and IIA metals and ammonia, sugars, urea and water-soluble urea derivatives, such as allantoin. Those skilled in the art will recognize that the foregoing materials can be commercially obtained in varying forms ranging from rock salt crystals to free flowing granules or powders.

Dead Sea salts are generally heterogeneous mixtures of crystalline and powder mineral salts obtained from the Dead Sea in Israel. A preferred exemplary Dead Sea salt is commercially sold by the Dead Sea Works Ltd., Beer-Sheva, Israel, and reportedly has the following typical composition:

| | |
|---|---|
| Potassium Chloride | 22-28% |
| Sodium Chloride | 8-18% |
| Calcium Chloride | 0.3-0.7% |
| Magnesium Chloride | 30-34% |
| Water of Crystallization | 26-30% |
| Bromides | 0.2-0.4% |
| Sulfates | 0.1-0.2% |
| Insolubles | 0.2-0.9% |

Exemplary granular, water-soluble salts of Group IA and IIA metals and ammonia preferably include, without being limited thereto, sodium chloride, potassium chloride, magnesium chloride, calcium chloride, ammonium chloride, sodium sulfate, potassium sulfate, magnesium sulfate, sodium nitrate, potassium nitrate, magnesium nitrate, calcium nitrate, sodium carbonate, potassium carbonate, sodium bicarbonate, potassium bicarbonate, ammonium bromide, sodium bromide, calcium bromide and the like and mixtures thereof.

Exemplary granular sugars include, without being limited thereto, cane sugars, beet sugars, water-soluble saccharides and polysaccharides.

Urea and highly water-soluble urea derivatives can be obtained in prill or pellet form and milled to a granular, free-flowing powder, if desired.

Additional benefits can be obtained with the anhydrous skin cleansers of this invention, by incorporating water-soluble abrasive ingredients that have high exothermic or endothermic heats of solution, such as magnesium chloride, ammonium chloride, magnesium sulfate and the like, in sufficient amounts to produce a discernible cooling or warming sensation to the touch during use. As small amounts of water are added while continuing to rub the product on the skin, depending on whether the heat of solution of the abrasive is endothermic or exothermic, formulations can be made which become cooler or warmer to the touch. Aside from the pleasurable sensations that can be achieved with cooling or warming, the warming can augment and enhance the cleaning process.

Optionally auxiliary cosmetic adjuvant ingredients known in the art can be employed, such as coloring agents, fragrances, preservatives, antioxidants, keratolytic agents, pH adjusting agents, and the like.

The anhydrous skin cleanser compositions of this invention can be prepared by heating together the emollient water-immiscible oil ingredients and emulsifier ingredients to a temperature of about 70° C. with sufficient mixing agitation to dissolve the emulsifier in the oil to provide a substantially anhydrous oil-emulsifier phase. The oil-gelling agent is then dispersed with mixing agitation in the anhydrous oil-emulsifier phase until a substantially non-runny, thickened gel phase begins to form, cooling the admixture, if necessary. The water-soluble abrasive ingredient, and optional ingredients, if any, are then added to the gel phase with sufficient mixing agitation to evenly disperse the ingredients in the gel phase. Those skilled in the art will understand that the order of incorporation of ingredients and temperatures employed may vary with the type of ingredient and the manner of dissolution recommended by the supplier of the material, such as in the case of commercially pre-gelled clay ingredients.

A preferred method embodiment for using substantially anhydrous skin cleansers formulated according to the invention comprises the following steps.

a. Applying the anhydrous skin cleanser to substantially dry (not wet) skin, preferably by manually rubbing the applied amount over the skin to thoroughly coat the skin. The rubbing action preferably is a gentle rubbing or massaging for a period of at least about 5 seconds, preferably about 5 to about 30 seconds, to promote the removal of oily or greasy, water-insoluble soils and skin cell residues.

b. Contacting the skin cleanser coated skin gradually with an amount of water sufficient to moisten the coated skin but not more than about twice the amount of anhydrous skin cleanser originally applied to the skin, further continuously rubbing and massaging the so-moistened skin until an emulsion visibly forms in situ on the skin, and the abrasive ingredient particles substantially dissolve. The formation of an emulsion in situ, on the skin can be observed as a change in the appearance of the substantially transparent or translucent coating to a substantially turbid (i.e., milky to opaque cream) liquid emulsion.

c. Removing the resultant cleanser emulsion from the cleansed skin, preferably by rinsing it off with water. The skin can then be dried.

Step “b” is preferably performed by wetting the fingers with tap water at ambient room temperature and massaging the skin cleanser coated skin with the wet fingers, and incrementally adding further small amounts of water in the same manner as desired. Alternatively, a measured amount of water can be applied, if desired. The total weight amount of water added is preferably in a weight range of about 0.1 to not more than about 2 times the amount of skin cleanser formulation applied to the skin. Dissolution of the abrasive ingredients can be determined during step “b” by a change in the tactile characteristic of the skin cleanser on the skin from grainy to smooth.

It is recognized that in Step “c” the emulsion can also be wiped off first and the wiped skin can be then rinsed. Preferably, the novel skin cleansers are readily and conveniently removable from the skin by rinsing with water, thereby eliminating the need for tissues or towels and avoiding disposal or laundry problems attendant with the use thereof.

Anhydrous skin cleanser formulations prepared with the ingredients and ranges proposed above have been found to possess a surprising combination of beneficial effects when used on soiled skin. The term “soil” as applied to skin includes soils from natural sources, such as cellular debris present on the skin, and soils from external sources topically applied to the skin, such as from makeup, cosmetics, industrial greases, and environmental dirt. The anhydrous skin cleansers thoroughly cleanse the skin of makeups and other water-immiscible cosmetic residues. They effectively remove various types of oily and greasy soils from the skin surface and difficult to solubilize residues. The anhydrous skin cleansers remove rough and dry skin cells. Moreover, the anhydrous skin cleansers smooth and polish the skin surface and leave the cleansed skin unusually soft and moisturized.

The mechanism of how the anhydrous skin cleanser cleanses the skin is not fully understood. On application of the anhydrous skin cleanser to the skin, it is believed that the combination of emollient oils helps dissolve oil-soluble greases and other makeup and cosmetic residues as well as removing environmental soils. The abrasives assist in scrubbing off non-dissolved dirt and rough dead skin cells. It is also believed that upon addition of a small amount of water the low HLB emulsifier initially allows the formulation to take in water to form a water-in-oil or multiphase emulsion, which assists in solubilizing additional soils off the skin while also removing water-soluble residues. As the abrasive particles begin to dissolve and the particle size becomes smaller, it is believed that the decreasing particle size serves to act as a finer and finer abrasive agent that assists in smoothing, polishing and buffing the now cleansed surface of the skin. Finally, after all the abrasive has dissolved and the skin cleanser water-in-oil emulsion is rinsed from the skin, the high HLB emulsifier assists in either forming a multiphase emulsion, such as a water-in oil-in-water emulsion or in inverting the emulsion to an oil-in-water emulsion, which helps in rinsing the soil containing skin cleanser from the skin.

The following Examples further illustrate the anhydrous skin cleansers of this invention with generally preferred embodiments, ingredients and methods, but are not intended to be limiting. Except where otherwise indicated, the INCI name of the ingredient is used.

### EXAMPLE 1

The following example illustrates the beneficial efficacy of practicing the method of this invention with the novel skin cleanser of this invention as a body cleanser and smoother.

| Ingredient | Weight % |
|---|---|
| Glyceryl stearate | 2.1 |
| PEG-100 stearate | 2.1 |
| Mineral oil | 60.9 |
| Fumed Silica | 4 |
| Dead Sea Salt | 30.4 |
| Fragrance | 0.5 |

The anhydrous skin cleanser was applied to body skin that was soiled with both oily and water-soluble materials by rubbing the anhydrous skin cleanser onto the skin with a massaging motion for about 10 seconds so that the skin was thoroughly coated. The coated skin was then moistened with a small amount of water by wetting the fingers of one hand and rubbing the skin with the wetted fingers until the anhydrous skin cleanser coating formed a white cream emulsion visible on the skin and the abrasive Dead Sea salt dissolved (i.e., salt crystals were no longer detected to the touch or visible in the emulsion). The so formed skin cleanser emulsion was then removed from the skin by rinsing with tap water and the cleansed skin dried. The dry cleansed skin was visually very clean, smooth and soft with no trace of harsh dryness normally associated with the use of abrasive cleaners.

For comparison the anhydrous skin cleanser formulation was applied following the procedure described above, except that the step of moistening the coating was omitted and the skin cleanser was rinsed off the skin with running tap water and then dried. Although the skin was clean and felt soft, smooth, and moisturized, the efficacy was judged overall to be less than that achieved when it was applied in the manner described above.

### EXAMPLE 2

This example illustrates an anhydrous skin cleanser gel suitable for use as a facial skin cleanser.

| Ingredient | Weight % |
|---|---|
| Mineral oil | 39.5 |
| Isopropyl stearate | 17.5 |
| Quaternium 18 Hectorite | 2.4 |
| Glyceryl stearate | 1.6 |
| PEG-100 stearate | 1.3 |
| Propylene carbonate | 0.7 |
| Cane Sugar (Refined) | 36.4 |
| Fragrance | 0.6 |

The anhydrous skin cleanser gel was used for facial cleansing and moisturizing by hand applying the skin cleanser onto the face and gently massaging the anhydrous skin cleanser over the face for several seconds. The palms of both hands were then slightly moistened with water and the massaging process continued. The process of moistening the palms and massaging was repeated until the sugar crystals could no longer be felt. The face was then rinsed and dried. Makeup and other facial soils had been removed and the face felt unusually soft, smooth and moisturized.

### EXAMPLE 3

This example illustrates an anhydrous skin cleanser gel suitable for use as a hand cleanser.

| Ingredient | Weight % |
|---|---|
| Mineral oil | 40 |
| Caprylic/Capric triglyceride | 40 |
| Glyceryl stearate | 3 |
| Steareth-100 | 2 |
| Aluminum/Magnesium | 7 |
| Hydroxide Stearate | |
| Sucrose | 7.5 |
| Fragrance | 0.5 |

The anhydrous skin cleanser gel was applied on hands that were soiled with a greasy automotive residue. The skin was then moistened with a small amount of water and rubbed until all the grittiness of the crystalline sucrose could no longer be felt. The cleanser was then rinsed from the skin with water. The cleansed hands were clean and had a pleasing soft feel.

### EXAMPLE 4

This example illustrates an anhydrous skin cleanser having the additional benefit of imparting a cooling effect on the skin.

| Ingredient | Weight % |
|---|---|
| Caprylic/Capric triglyceride | 20 |
| Lanolin oil | 3 |
| Octyl stearate | 19.4 |
| Wheat Germ oil | 14.5 |
| Avocado oil | 6.3 |
| Aluminum/Magnesium hydroxide | 4 |
| stearate | |
| Sorbitan stearate | 3 |
| PEG-40 stearate | 2 |
| Ammonium chloride | 27 |
| Antioxidant | 0.1 |
| Fragrance | 0.7 |
| Color | qs |

The anhydrous skin cleanser was applied and rubbed onto the skin of the forearm, a small amount of water was added and rubbing continued. The cleanser coated skin became noticeably cooler. Additional quantities of water were incrementally added and rubbing continued until all the ammonium chloride crystals had dissolved and an emulsion had formed on the skin. The emulsion cleanser was then rinsed from the skin with water. The skin-was clean, soft and smooth.

### EXAMPLE 5

This example illustrates an anhydrous skin cleanser having a cooling effect on the skin.

| Ingredient | Weight % |
|---|---|
| Isopropyl palmitate | 15 |
| Lanolin oil | 15 |
| Wheat Germ oil | 15 |
| Isohexadecane | 15 |
| Glyceryl stearate | 3 |
| Polysorbate-20 | 2 |
| Magnesium stearate | 10 |
| Aluminum stearate | 10 |
| Ammonium chloride | 15 |

The skin cleanser was applied to soiled skin that was judged rough and dry and rubbed into the skin. After rubbing for about 30 seconds, the skin was moistened with water and rubbing the cleanser coated skin was resumed until an emulsion formed on the skin. The cleanser emulsion was rinsed from the skin with water and the cleansed skin dried. The skin was judged as having a lubricous smooth feel, and much of the roughness had disappeared.

### EXAMPLE 6

This example illustrates an anhydrous skin cleanser suitable for use on the feet.

| Ingredient | Weight % |
|---|---|
| Mineral oil | 25.5 |
| Octyl stearate | 15.5 |
| Isododecane | 21 |
| Steareth-2 | 3 |
| Steareth-20 | 1 |
| Fumed Silica | 4.6 |
| Sodium chloride | 28.5 |
| Fragrance | 0.9 |

The anhydrous skin cleanser gel was applied and massaged onto the skin of one foot. After massaging for about 20 seconds, one hand was moistened with water and the massaging continued with the water wet hand. The process of moistening the hand and continuing massaging was repeated until the abrasive salt crystals had dissolved sufficiently so that they could no longer be felt and the cleanser had formed an emulsion on the skin. The cleanser emulsion was then rinsed from the foot with water and dried. The cleansed, treated foot was judged to be considerably softer, smoother and had less rough dead skin cells on its surface than the untreated foot.

### EXAMPLE 7

This examples illustrates a self-warming anhydrous skin cleanser gel.

| Ingredient | Weight % |
|---|---|
| Soya oil | 17 |
| Octyl palmitate | 45.5 |
| Glyceryl stearate | 3 |
| Steareth-100 | 2 |
| Magnesium sulfate | 29.3 |
| Stearalkonium Hectorite | 2.5 |
| Propylene carbonate | 0.7 |

The anhydrous skin cleanser gel was first rubbed onto rough dry skin and then a small amount of water was added with continued rubbing. The skin cleanser began to form a cream as water was taken into the oil-based gel and a pronounced discernible warming effect was noted as heat was liberated by the dissolving magnesium sulfate. The process was continued until the abrasive magnesium sulfate crystals could no longer be felt and an emulsion had formed on the skin. The cleanser emulsion was then rinsed off the skin with water and the skin dried. The rough dead skin cells had been removed and the skin felt soft and moisturized.

The amount of warming generated by Formula 7 was measured by placing 10 grams of the anhydrous skin cleanser gel into an insulated cup, then adding 5 grams of water and mixing. The mixture was continuously stirred to form an emulsion and the rise in temperature measured. The temperature of the added water was about 15° C. and the temperature of the mixture, within one minute, increased to about 53° C.

### EXAMPLE 8

This example illustrates an anhydrous skin cleanser gel suitable for use as an in-shower, all-in-one, body cleanser, scrub, polisher and moisturizer.

| Ingredient | Weight % |
|---|---|
| Mineral oil | 28.5 |
| Caprylic/Capric triglyceride | 9 |
| Isohexadecane | 18.5 |
| Sorbitan stearate | 3.5 |
| Polysorbate-20 | 1.5 |
| Magnesium stearate | 4 |
| Aluminum Stearate | 4 |
| Dead Sea Salt | 31 |

The skin cleanser gel was used as follows. Before becoming wet in the shower, the skin cleanser gel was hand applied all over the body and massaged for about one minute. Both hands were then moistened in the stream of the shower and the body massaging process continued. The process of wetting the hands and continuing to massage was continued until all the Dead Sea Salt had dissolved and abrasive particles could no longer be felt and an emulsion had formed. The cleanser emulsion product was then rinsed off the body in the shower. After drying, the skin had an unusually clean and fresh appearance. It felt smooth, soft and appeared as if a moisturizing agent had been applied.

### EXAMPLE 9

This example illustrates an anhydrous skin cleanser gel suitable for use as a rough skin cleanser and smoother.

| Ingredient | Weight % |
|---|---|
| Sunflower oil | 40 |
| Soy oil | 37 |
| Disteardimonium Hectorite | 5 |
| Propylene carbonate | 1.5 |
| Laureth-1 | 2.8 |
| Laureth-23 | 2 |
| Urea | 11.2 |
| Tocopherol | 0.1 |
| Fragrance | 0.4 |

The anhydrous skin cleanser gel was applied and rubbed onto rough dry skin of the knee for about 10 seconds. A small amount of water was added by moistening the hand and the rubbing continued with the moistened hand. Another small amount of water was similarly added while continuing to rub the product onto the knee. After an emulsion had formed on the skin of the knee and no further grittiness could be felt, the cleanser emulsion product was rinsed off with water and the knee dried. The cleansed skin on the knee was much smoother and softer than before the cleansing treatment.

### EXAMPLE 10

This example illustrates an anhydrous skin cleanser gel suitable for use as a hand cleanser.

| Ingredient | Weight % |
|---|---|
| Avocado oil | 25 |
| Coconut oil | 25 |
| Jojoba oil | 25 |
| Steareth-2 | 3 |
| Steareth-20 | 1 |
| Fumed Silica | 4 |
| Sodium chloride | 17 |

The anhydrous skin cleanser gel was applied and rubbed onto rough dry hands that were heavily soiled with greasy residue. A few drops of water were then added and the skin cleaner continued to be rubbed in until the gritty feel of the salt disappeared as it dissolved and an emulsion formed on the skin. The hands were then rinsed with water and dried. The oily soil was completely removed and the hands were left with a soft, very smooth feel.

The present invention has been described generally and with respect to preferred embodiments. It will be understood that modifications and variations of the disclosed method, compositions and delivery system may be made without departing from the spirit and scope of the novel concept of the present invention.

We claim:

1. A substantially anhydrous skin cleanser gel comprising:
a) a water-immiscible emollient oil;
b) an oil-gelling agent that is insoluble in water and in oil;
c) an emulsifying agent capable of forming an emulsion in situ on skin during use; and
d) a substantially crystalline abrasive material that is soluble in water but insoluble in the resulting anhydrous skin cleanser gel.

2. The anhydrous skin cleanser gel of claim 1, wherein the weight percent amount of water-immiscible emollient oil, on a total composition weight basis, is in the range of about 30% to about 90%.

3. The anhydrous skin cleanser gel of claim 1 wherein the weight percent amount of water-immiscible emollient oil, on a total composition weight basis, is in the range of about 40% to about 85%.

4. The anhydrous skin cleanser gel of claim 1, wherein the weight percent amount of oil-gelling agent, on a total composition weight basis, is in the range of about 1% to about 20%.

5. The anhydrous skin cleanser gel of claim 1, wherein the weight percent amount of oil-gelling agent, on a total composition weight basis, is in the range of about 2% to about 10%.

6. The anhydrous skin cleanser gel of claim 1, wherein the weight percent amount of emulsifying agent, on a total composition weight basis, is in the range of about 1% to about 15%.

7. The anhydrous skin cleanser gel of claim 1, wherein the weight percent amount of emulsifying agent, on a total composition weight basis, is in the range of about 2% to about 10%.

8. The anhydrous skin cleanser gel of claim 1, wherein the weight percent amount of abrasive material, on a total composition basis, is in the range of about 1% to about 60%.

9. The anhydrous skin cleanser gel of claim 1, wherein the weight percent amount of abrasive material, on a total composition basis, is in the range of about 2% to about 50%.

10. The anhydrous skin cleanser gel of claim 1 wherein the water-immiscible emollient oil is selected from the group consisting of linear and branched chain aliphatic hydrocarbons, animal derived oils, vegetable derived oils, silicone oils, esters of fatty acids having at least 7 carbon atoms in the fatty chain, C₇-C₁₈ aliphatic and aromatic alcohols, and mixtures thereof.

11. The skin cleanser gel of claim 1 wherein the oil-gelling agent is selected from the group consisting of clays, organically modified clays, silicas, soaps of fatty acids having at least 11 carbon atoms in the fatty chain and a Group IA, Group IIA or Group IIIA metal ion, aluminum/magnesium stearate gels and mixtures thereof.

12. The skin cleanser gel of claim 1 wherein the emulsifying agent is selected from the group consisting of ethoxylated carboxylic acids, ethoxylated glycerides, glycol esters and derivatives thereof, monoglycerides, polyglyceryl esters, polyhydric alcohol esters and ethers, sorbitan/sorbitol esters, triesters of phosphoric acid, ethoxylated fatty alcohols, propoxylated polyoxyethylene (POE) ethers and mixtures thereof.

13. The skin cleanser gel of claim 1 wherein the emulsifying agent comprises at least one emulsifying agent having a reported HLB value of not more than 8.

14. The skin cleanser gel of claim 13 further including at least one emulsifying agent having a reported HLE value of at least 8.

15. The skin cleanser gel of claim 1 wherein at least one emulsifying agent has a reported HLB value in the range of about 3 to about 7 and at least one emulsifying agent has a reported HLB value in the range of about 10 to about 18.

16. The anhydrous skin cleanser of claim 1 wherein the abrasive material is selected from the group consisting of Dead Sea salt, salts of Group IA and IIA metals and ammonia, sugars, urea and water-soluble derivatives thereof, and mixtures thereof.

17. The anhydrous skin cleanser of claim 1 further containing cosmetic adjuvants selected from the group consisting of coloring, fragrances, preservatives, antioxidants, keratolytic agents, pH adjusting agents, and mixtures thereof.

18. The anhydrous skin cleanser of claim 1 wherein the abrasive material has an exothermic heat of solution in water.

19. The anhydrous skin cleanser of claim 1 wherein the abrasive material has an endothermic heat of solution in water.

20. The anhydrous skin cleanser of claim 1 wherein the abrasive material has a minimum solubility of 20 parts by weight in 100 parts by weight of water at temperature of about 20° C. and a particle size in the range of about 50 to about 1000 microns.

21. The anhydrous skin cleanser of claim 1 wherein the abrasive material is Dead Sea salt.

22. A substantially anhydrous skin cleanser gel comprising on a total compositions weight basis:
a) about 30 to about 90% by weight of water-immiscible emollient oil;
b) about 1% to about 20% of oil-gelling agent that is insoluble in water and in oil;
c) about 1% to about 15% of emulsifying agent capable of forming an emulsion in situ on skin during use; and
d) about 1% to about 60% of substantially crystalline abrasive material that is soluble in water but insoluble in the anhydrous skin cleanser gel.

23. The skin cleanser gel of claim 22 wherein the emulsifying agent comprises at least one emulsifying agent having a reported HLB value of not more than 8.

24. The skin cleanser gel of claim 23 wherein the emulsifying agent further comprises at least one emulsifying agent having a reported HLB value of at least 8.

25. The skin cleanser gel of claim 24 wherein when the skin cleanser gel is applied to the skin and coated thereon, an emulsion forms in situ on contacting the so coated skin with an amount of water of not more than about twice the amount of skin cleanser gel originally applied.

26. A method of cleansing the skin with the anhydrous skin cleanser gel of claim 1 comprising the steps of;
a. applying anhydrous skin cleanser gel to substantially dry skin, as by manually rubbing the applied amount over the skin to coat the skin for at least about 5 seconds;
b. contacting the skin cleanser coated skin with an amount of water sufficient to moisten the coated skin, the amount of water being not more than about twice the amount of anhydrous skin cleanser originally applied to the skin, and further continuously rubbing and massaging the moistened skin until the cleanser forms an emulsion in situ on the skin, and the abrasive ingredient particles substantially dissolve; and
c. removing the so-formed cleanser emulsion from the cleansed skin, as by rinsing with water.

27. A method of cleansing the skin with the anhydrous skin cleanser gel of claim 22 comprising the steps of;
a. applying anhydrous skin cleanser to substantially dry skin, as by manually rubbing the applied amount over the skin to coat the skin for at least about 5 seconds;
b. contacting the skin cleanser coated skin with an amount of water sufficient to moisten the coated skin, the amount of water being not more than about twice the amount of anhydrous skin cleanser originally applied to the skin, and further continuously rubbing and massaging the moistened skin until the cleanser forms an emulsion in situ on the skin, and the abrasive ingredient particles substantially dissolve; and
c. removing the so-formed cleanser emulsion from the cleansed skin, as by rinsing with water.

28. The anhydrous skin cleanser of claim 1 wherein the anhydrous skin cleanser gel is capable of forming an emulsion in situ on skin coated with the anhydrous skin cleanser, when the so-coated skin is gradually contacted with water, the resultant emulsion being removable from the skin with water.

29. The anhydrous skin cleanser of claim 22 wherein the anhydrous skin cleanser gel is capable of forming an emulsion in situ on skin coated with the anhydrous skin cleanser, when the so-coated skin is gradually contacted with water, the resultant emulsion being removable from the skin with water.
